(19) United States
(12) Patent Application Publication
(10) Pub. No.: US 2024/0164662 A1
(43) Pub. Date: May 23, 2024

(54) DEVICE AND METHOD FOR DETECTING A MOVEMENT OR STOPPING OF A MOVEMENT OF A PERSON OR OF AN OBJECT IN A ROOM, OR AN EVENT RELATING TO SAID PERSON

(71) Applicant: KAPCARE SA, Brussels (BE)

(72) Inventor: Philippe Kaplan, Brussels (BE)

(21) Appl. No.: 18/283,935

(22) PCT Filed: Feb. 25, 2022
(86) PCT No.: PCT/EP2022/054862

§ 371 Date: Sep. 25, 2023

(30) Foreign Application Priority Data
Mar. 26, 2021 (EP) 21165410
Jul. 6, 2021 (EP) 21183963

(51) Int. Cl. A61B 5/11

(52) U.S. Cl. CPC A61B 5/1113; A61B 5/1117

(57) ABSTRACT

The present invention relates to a device and a method for detecting whether a person or an object located in a room has performed a movement or has interrupted a movement, and being able to deduce therefrom whether an event has happened to said person like, for example, that he/she has left an area of the room or that he/she has returned to it after having left it. The device performs an analysis over time of the distances between the sensors of a detector and the obstacles present in the detection field of the detector. The detection of a movement or of the interruption of a movement is based on the analysis of the variations of said distances over time. The deduction of an event is based on the location of the obstacles moving within a space of the room and on the history of the events that have occurred to said person within the room.

### FIELD OF THE INVENTION

The invention relates to the field of devices for monitoring people and or objects, in particular to devices and methods allowing determining events relating to a person.

### PRIOR ART

The detection of some events occurring to a person placed under monitoring is a major concern, in particular in hospital centres and retirement homes. For example, this detection is important when the person is subject to particular conditions or circumstances, such as an illness (in particular dementia, Alzheimer, . . . ), a post-operative situation, or a history of falls. Thus, for example, it is important to detect that a person leaves his/her bed voluntarily or that he/she has fallen thereof or that he/she does not return to his/her bed within a reasonable time. This monitoring is particularly sought after in care units, such as hospitals, care clinics or retirement homes, in order to prevent any deterioration in the condition of patients, although this monitoring could also be done in other environments.

The rhythm within care units prevents qualified personnel from being able to monitor all bedridden persons to detect these events.

Thus, different devices have been developed to detect them. With such devices, a patient leaving the bed, and a fortiori the patient falling, normally triggers an alert to the attention of the hospital personnel. For example, mention may be made of the device and the method for detecting getting out of bed and for detecting falling disclosed in the document EP3687379A1. The device disclosed in the document EP3687379A1 comprises a detector having a detection field able to cover at least one portion of the bed and at least one portion of its environment, said detector including several distance sensors, each distance sensor being able to provide distance measurements over time between said sensor and a corresponding obstacle in its line of sight, said device further comprising a processing unit connected to the detector and configured to process the distance measurements provided by the distance sensors of the detector.

Responding to alerts issued by such devices monopolises qualified personnel who are no longer available to care for other patients. Yet, some bed leaves take place without putting the patient in danger, like for example going to the toilet and then returning to bed. In such a scenario, there is no need to alert the hospital personnel.

The device and the method disclosed in the document EP3687379A1 do not allow detecting a possible return to bed and could in some cases unnecessarily trigger alarms.

### SUMMARY OF THE INVENTION

The invention aims to provide a device and a method allowing determining whether a person in a room has performed a movement or whether an event relating to this person has happened. In particular, the device and the method according to the invention allow detecting with relative assurance that a person having left a first volume of the room and not having fallen has returned there, which allows avoiding triggering of an alarm if this return takes place within a predefined period. For example, this could consist in leaving a bed and returning to bed. It could also consist of be a person seated in an armchair or occupying a given portion of the room.

The invention is defined by the independent claims. The dependent claims define preferred embodiments of the invention.

According to the present invention, a device is provided for detecting a movement or a stop of a movement of a person or an object in a room, or an event relating to said person, the device comprising:
a detector having a detection field able to cover at least one first volume of said room and at least one portion of the environment of the first volume, said detector including several distance sensors, each distance sensor having a line of sight and being able to provide distance measurements over time between said sensor and a corresponding obstacle, i.e. an obstacle located in the line of sight of said sensor,
a processing unit connected to the detector and configured to process over time the distance measurements received from the distance sensors of the detector,
   characterised in that the processing unit is configured to perform the following steps and in that order:
a. for each of the distance sensors of a first set of distance sensors of the detector, determining a first corresponding reference distance either as a distance measured at a first time point (t₁) by the corresponding distance sensor, or as being a combination of distances measured at several first time points (t₁, t₂, t₃, t₄) by the corresponding distance sensor,
b. determining a second set of distance sensors which consists of those of the distance sensors of the first set including a distance measurement performed at a time point (t₅) subsequent to the first time point (t₁) or to the first time points (t₁, t₂, t₃, t₄) differs by more than a predetermined first value from the first reference distance of the corresponding distance sensor,
c. selecting at least one first part of the distance sensors of the second set of distance sensors and associating said subsequent time point (t₅) with the at least one first part of distance sensors,
d. determining, for the selected at least one first part of distance sensors, a first representative position of the positions in space of the obstacles corresponding to the distance sensors of said selected at least one first part of sensors, and associating the subsequent time point (t₅) with said first representative position,
e. repeating steps a. to d. at several other time points (t₆, t₇, t₈) subsequent to the first time point (t₁) or to the first time points (t₁, t₂, t₃, t₄),
f. associating representative positions associated with the several different subsequent time points (t₅, t₆, t₇, t₈) to form a first association of representative positions,
g. selecting, within the first association of representative positions, at least one first pair of representative positions and calculating a first speed as the distance between the representative positions of said first pair divided by the duration separating the subsequent time points (t₅, t₆) associated with the representative positions of said first pair,
h. deciding, when said first speed is higher than a predetermined second value, that the person or the object has performed a movement in the room between the subsequent time points (t₅, t₆) associated with the representative positions of said first pair of representative positions.

With such a device, it is possible to identify a movement in the detection field of the detector, and in particular a movement with a given magnitude. Such a movement could be leaving bed, falling, or returning to bed by a person.

Preferably, the combination of the distances measured at the first time points t₂, t₃, t₄) is an average of the distances measured at the first time points (t₁, t₂, t₃, t₄). Indeed, with such a combination of the distance measurements, the reference distance of a sensor is the result of low-pass filtering over time of some of its measurements, which allows obtaining a better contrast of the last distance measurement.

Advantageously, said first representative position is the position of the geometric centre of the obstacles corresponding to said selected at least one first part of distance sensors.

Indeed, the geometric centre could be likened to the centre of mass of an object or of a person, its movement represents an average of the movements of the parts of the object or of the person and is therefore more representative of the movement of the object or of the person over time.

Advantageously, the processing unit is configured to:
form at least one association of representative positions and select, within each of the formed associations of representative positions, at least one pair of representative positions and calculate, for each of the selected pairs of representative positions, a speed as being the distance between the representative positions of the selected pair divided by the duration separating the subsequent time points (t₅, t₆) associated with the representative positions of said pair,
determine whether all of the speeds calculated for the selected pairs of representative positions are lower than the predetermined second value,
decide, if so is the case, that the person or the object has stopped all movements.

Indeed, if a movement of the person has been identified, it is appropriate to detect when this movement has stopped in order to have information better enabling the detection of events relating to the person.

More advantageously, the speeds are calculated for selected pairs of representative positions forming part of the same association of pairs of representative positions formed by pairing, for example, each representative position forming part of said association with a representative position of said association which directly follows it chronologically, and only with the latter. Thus, the device is provided with a continuous measurement of the speed of the person or of an object moved by the person.

Advantageously, the processing unit is configured to:
determine whether the first representative position is located in the first volume or in a second volume or in a third volume in space, the first volume being a volume extending vertically upwards and/or downwards from a first horizontal surface of said room, the second volume being a finite volume extending outwards from the lateral limits of the first volume, the third volume being a volume extending outwards from the lateral limits of the second volume,
decide that said person or object was located in that one amongst the first, second or third volume where said first representative position is located at the subsequent time point associated therewith (t₅).

Indeed, the knowledge of the location of the person in the room at a given time point allows, together with other information, deducing with greater confidence whether a given event relating to the person has occurred.

Preferably, the processing unit is configured to determine, or to interrogate another processing unit to know whether said person is in the first volume, has left the first volume or has fallen after having left the first volume.

Indeed, the knowledge of the last event relating to the person allows discarding some possible future events.

Preferably, the processing unit is configured to verify at a given time point whether:
said person has left the first volume and has not fallen,
said person or object has performed a movement in the room,
said person or object was located in the first volume at the time of said movement,
   and to decide that said person has returned to the first volume if all of these conditions are met simultaneously.

Indeed, by cross-referencing different types of information relating to the person, it is possible to deduce the occurrence of a particular event relating to him/her with greater confidence.

Preferably, the processing unit is configured to verify at a given time point whether:
said person has left the first volume and has not fallen,
said person or object has stopped all movements,
said person or object was last located in the first volume or in the second volume,
   and to decide that said person has returned to the first volume if all of these conditions are met simultaneously.

Indeed, by cross-referencing different types of information relating to the person, it is possible to deduce the occurrence of a particular event relating to him/her with greater confidence.

Advantageously, the processing unit is configured to verify at a given time point whether:
said person or an object has performed a movement,
said person was in bed before said movement,
said person or an object was located in the second volume at the time of said movement,
and to decide that said person has left the first volume if all of these conditions are met simultaneously.

Indeed, by cross-referencing different types of information relating to the person, it is possible to deduce the occurrence of a particular event relating to him/her with greater confidence.

The present invention also relates to a method for detecting a movement or a stop of a movement of a person or an object in a room, or an event relating to said person.

### BRIEF DESCRIPTION OF THE FIGURES

These aspects as well as other aspects of the invention will be clarified in the detailed description of particular embodiments of the invention, reference being made to the drawings of the figures, wherein:

FIG. 1 schematically shows an overview of a device according to the invention in the context of a room;

FIG. 1b is schematically shows a top view of the chamber of FIG. 1;

FIG. 2 schematically shows part of a processing performed by the device according to the invention;

FIG. 3 schematically shows part of a processing performed by the device according to a preferred embodiment of the invention;

The drawings of the figures are neither to scale nor proportionate. In general, similar or identical sensors are denoted by identical numerals in the figures.

### DETAILED DESCRIPTION OF EMBODIMENTS OF THE INVENTION

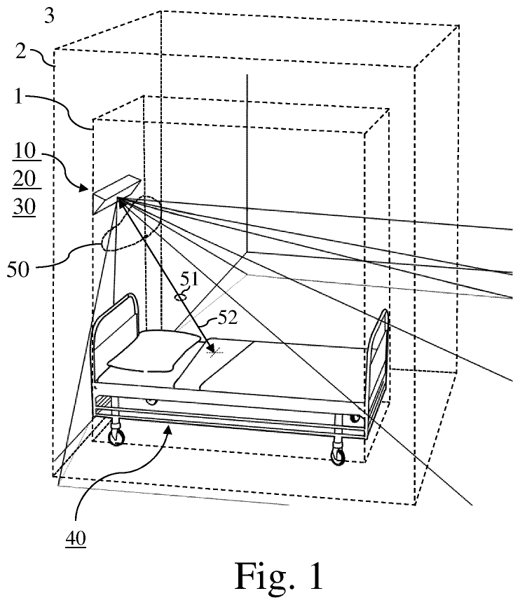

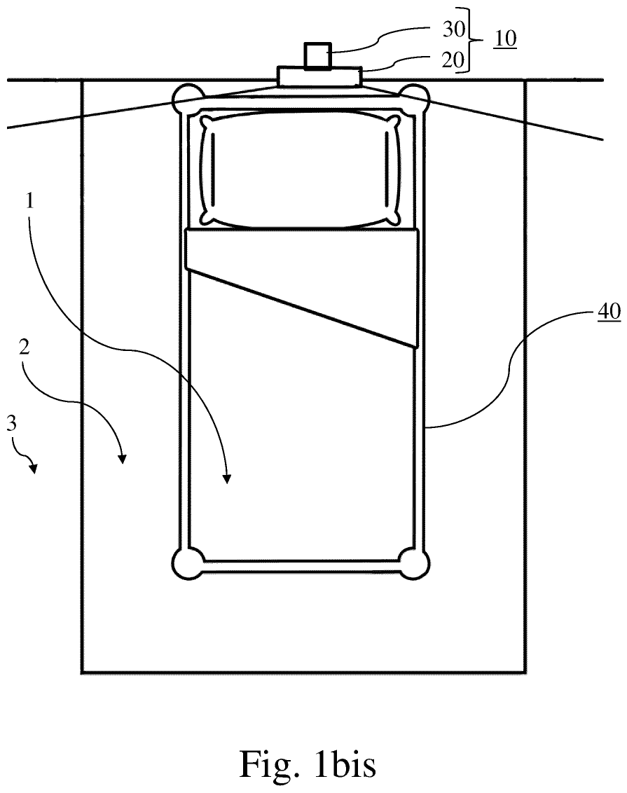

FIG. 1 schematically shows an overview of a device (10) according to the invention when it is mounted for example in a room in which a bed (40) is installed on which a person could be bedridden (the person is not shown) whose movements should be monitored. Nonetheless, other application cases are also possible such as the monitoring of a collection item in a museum or any other case where it is necessary to be able to monitor the movement of a person or an object in a closed space.

The device (10) according to the invention comprises a detector (20) having a detection field (50) able to cover at least one portion of the bed (40) and at least one portion of the environment of the bed. The detection field of the detector is that part of space in which the detector is able to carry out its function. The detector (20) includes several distance sensors, each distance sensor having a line of sight (51) and being able to provide distance measurements (52) over time between said sensor and an obstacle corresponding to said sensor, i.e. an obstacle located in the line of sight (51) of said distance sensor. Hence, an obstacle corresponding to a distance sensor is that portion of an object or a person which is at the end of the line of sight of said distance sensor and it could, for ease of understanding, be assimilated to a point if the detection angle of the sensor is small, which is usually the case.

For example, the detector (20) may be a camera operating on the principle of time of flight (“Time of Flight” or “TOF”) and which allows measuring directly or indirectly and in real-time distances relative to an observed three-dimensional scene. To do so, a TOF camera illuminates the scene that lies in its detection field and calculates, for each distance sensor (sometimes also called “photosensitive element” or “pixel” in this context) of the camera, the time that the emitted light takes to travel between the distance sensor and its corresponding obstacle.

Since the speed of light is constant, this travel time is directly proportional to the distance between a distance sensor and its corresponding obstacle. This travel time measurement is performed independently for each distance sensor of the camera.

A concrete example of such a detector is the “SwissRanger 4000” or “SR4000” camera from MESA Imaging, which includes an array of 176×144 distance sensors (photosensitive elements).

Preferably, the detector (20) is placed or configured to be placed at a height with respect to the ground which is larger than the maximum height of the upper surface of said bed and it is oriented or designed to be oriented so that its detection field (50) covers at least one portion of the bed (40), preferably the entire bed, and at least one portion of its environment.

As an example, as illustrated in FIG. 1, the detector (20) may be placed against a wall above the head of the bed, but other positions may be considered, like for example against a wall opposite to that against which the bed is placed. The detector (20) may also be arranged so as to have in its detection field (50), on the one hand at least one portion of the bed, preferably the entire bed, and on the other hand a door allowing leaving and entering the room where the detector (20) is located.

The device (10) further comprises a processing unit (30) connected to the detector (20) and configured to acquire and process the distance measurements (or travel time, which is equivalent within a constant factor margin) provided by the distance sensors of the detector over time.

Preferably, the processing unit is able to memorise the different distance measurements provided by the distance sensors over time and to process at a given time point distance measurements taken and memorised at different time points.

The processing unit (30) may process these distance measurements periodically, aperiodically or continuously, in the latter case within the limits of the maximum rate at which the detector could provide the distance measurements. In the case of a periodic assessment, the processing unit (30) processes the distance measurements received for example every 5 seconds, or every 4 seconds, or every 3 seconds, or every 2 seconds, or every second, or every 0.5 seconds. In the case of an aperiodic assessment, the processing unit (30) may process the distance measurements received at time points selected for example according to the history of the received distance measurements.

The processing unit (30) may be any means allowing receiving and analysing the distance measurements provided by the detector (20), and that being so as a function of time. For example, it may consist of a microprocessor executing a program for analysing the data corresponding to the distance measurements provided by the detector.

Note that the processing unit (30) could be grouped together with the detector (20), for example within the same case. Alternatively, the detector (20) may be separated from the processing unit (30) and be provided with data communication means, preferably wireless, such as by WI-FI (IEEE 802.11 standard), enabling data transfer to the processing unit (30). The processing unit (30) then comprises means for receiving the data transmitted by the detector (20) via the data communication means. In this case, said data comprise at least the distance measurements or the times of flight provided by the distance sensors of the detector (20).

First of all, the processing unit (30) determines a first reference distance for each of the distance sensors of a first set of distance sensors of the detector (20). The first reference distance is either a distance measured at a first time point (t₁) by the corresponding distance sensor, or a combination of distances measured at several first time points (t₁, t₂, t₃, t₄) by the corresponding distance sensor. It should be noted that said first set may include all or part of the distance sensors of the detector.

In the case of a combination, the processing unit (30) therefore combines several distance measurements performed at different time points by the same sensor to create a first reference distance associated with said sensor. A first reference distance may be determined for all of the sensors of the detector (20) or only for part of these. Preferably, the processing unit (30) calculates a first reference distance for all of the sensors of the detector.

Similarly, the combination may be different for each sensor. Preferably, the processing unit (30) carries out the same combination of distance measurements for all sensors. Preferably, the processing unit (30) combines distance measurements received from the sensors over a period comprised between 0 minutes and 30 minutes, preferably for a period of 5 minutes. Each first reference distance may be updated by the processing unit (30) independently for each sensor, in particular at different time points depending on the sensor.

The first reference distances may be determined by means of distance measurements obtained at time points that are different for each sensor.

Preferably, the processing unit (30) uses, to determine a first reference distance, distance measurements received at the same time points for all the distance sensors of the detector.

Preferably, the processing unit (30) updates the first reference distances periodically using the respectively most recent distance measurement(s) received from the sensors.

Preferably, the processing unit (30) updates the first reference distance of a sensor each time a new distance measurement is received from said sensor.

Afterwards, the processing unit (30) determines a second set (60) of distance sensors which consists of those of the distance sensors of the first set including a distance measurement performed at a time point (t₅) subsequent to the first time point (t₁) or to the first time points (t₁, t₂, t₃, t₄) differs by more than a predetermined first value from the first reference distance of the corresponding distance sensor.

Preferably, the predetermined first value is comprised between 0 cm and 30 cm, more preferably between 0 and 20 cm, even more preferably between 0 and 10 cm. More preferably, the predetermined first value is 0 cm.

Afterwards, the processing unit (30) selects at least one part (61) of the sensors of the second set (60) and associates with the at least one part (61) the time point (t₅) (i.e. the time point when the sensors of the first set have performed distance measurements each having decreased or increased further by the predetermined first value with respect to their reference distances).

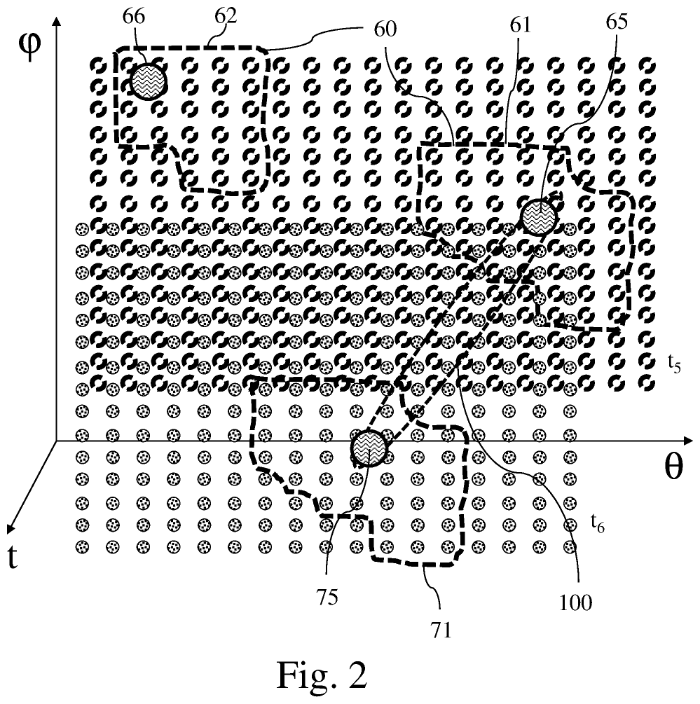

This aspect is illustrated in FIG. 2 which schematically shows different distance sensors of the detector (20) arranged according to an array whose indices correspond to the angular coordinates (in the case of a spherical coordinate reference frame) of the beams originating from these sensors, i.e. of their lines of sight (assumed to be rectilinear, as shown in FIG. 1), the origin of the spherical coordinate system could be taken for example on the detector (20). Hence, at each point of this array, it is possible to associate at a given time point the distance measured by the sensor corresponding to the indices of the point of the array. Hence, at each point in FIG. 2 also corresponds to an obstacle in the line of sight of the sensor represented by this point.

FIG. 2 shows an example of a second set (60) of distance sensors as defined hereinabove and comprising a first part (61) of distance sensors and a second part (62) of distance sensors at the time point (t₅).

FIG. 2 also illustrates a third part of distance sensors (71) which are those distance sensors whose distance measurement performed at a time point (t₆) differs by more than the predetermined first value of the reference distance of said distance sensor. In general, the reference distances that have led to the determination of a part of distance sensors should not be the same as the reference distances that have led to the determination of another part of distance sensors.

Preferably, the processing unit (30) groups together distance sensors having neighbouring coordinates (having neighbouring indices) of a set of distance sensors (60) within the same part of distance sensors (61).

Preferably, the union of all parts of distance sensors (61, 62) of the same set of distance sensors (60) associated with the same time point (t₅) includes all of the distance sensors of said set of distance sensors (60).

Afterwards, the processing unit (30) determines, at least for the selected first part (61) of distance sensors, a first representative position (65) of the positions in space of the obstacles corresponding to said selected first part of sensors (61), and associates the time point (t₅) associated with said first part of distance sensors (61). For example, such a first representative position (65) may be that of an obstacle corresponding to one of the sensors of the first part of distance sensors (61). In general, the first representative position (65) may be a combination of the positions in the space of the obstacles corresponding to part or all of the distance sensors of the first part of distance sensors (61). In a preferred embodiment of the invention, said first representative position (65) is the position of the geometric centre of the obstacles corresponding to said selected first part (61) of distance sensors. This geometric centre may be calculated by the processing unit starting from the spatial coordinates of said obstacles and therefore represents a point in space.

In a device according to the invention, the processing unit (30) is configured to associate representative positions (65, 75, 85, 95) associated with several different time points (t₅, t₆, t₇, t₆). In general, said representative positions (65, 75, 85, 95) are determined for parts of distance sensors (61, 71, 81, 91) which may be identical or different. Thus, FIG. 2 illustrates an association (100) of two representative positions (65, 75) determined for two parts of distance sensor (61, 71) associated with two different time points (t₅, t₆), said parts of distance sensor (61, 71) including generally different distance sensors. For practical reasons, FIG. 2 shows the two representative positions (65, 75) according to only their angular coordinates although a representative position (65, 75) is expressed by means of spatial coordinates. The processing unit (30) may be configured to associate within the same association (100) any number of representative positions (65, 75, 85, 95). The durations separating the time points (t₅, t₆, t₇, t₈) associated with representative positions (65, 75, 85, 95) may be arbitrary and in particular different.

Preferably, the processing unit (30) associates representative positions 65, 75, 85, 95) associated with time points (t₅, t₆, t₇, t₈) separated by a duration which can vary between 0.1 seconds and 300 seconds, preferably it is configured for a second duration.

Preferably, the processing unit (30) associates representative positions (65, 75, 85, 95) associated with the most recent time points.

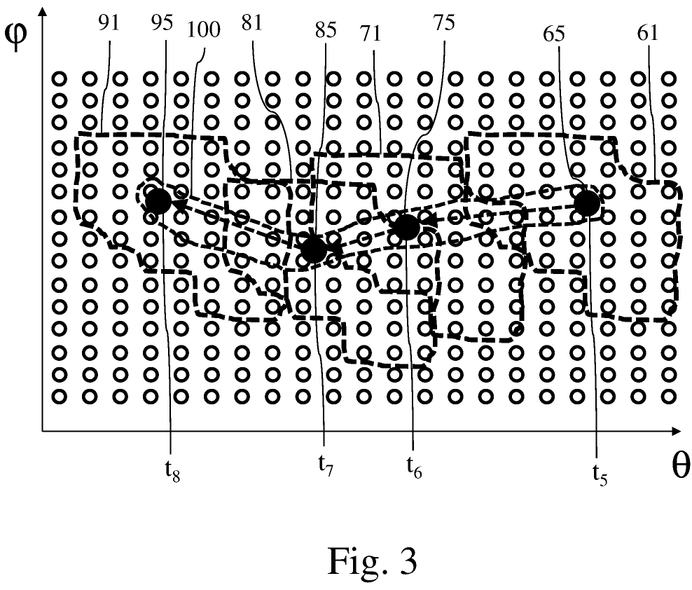

Preferably, the processing unit (30) associates representative positions (65, 75, 85, 95) each time new distance measurements are received from sensors. Preferably, representative positions that correspond to parts of distance sensors that have distance sensors in common are associated. FIG. 3 illustrates such an association (100) of four representative positions (65, 75, 85, 95) which correspond to four parts of distance sensors (the four areas surrounded by a dotted line: 61, 71, 81, 91) associated with four different time points (t₅, t₆, t₇, t₈) and having a given number of sensors in common (in the areas where the dotted lines overlap). One could see in FIG. 3 that a part of distance sensors (61, 71, 81, 91) of the association (100) of representative positions (65, 75, 85, 95) has at least one distance sensor in common with another part of distance sensors of the same association (100). Thus, in FIG. 3, the part (61) has three distance sensors in common with the part (71).

Afterwards, the processing unit (30) is configured to determine, within an association (100) of representative positions (65, 75, 85, 95), at least one pair of representative positions (65, 75) and to calculate for said at least one pair of representative positions (65, 75) a speed as being the distance between said representative positions (65, 75) of said pair, divided by the duration separating the time points (t₅, t₆) associated with said representative positions (65, 75).

In the example illustrated in FIG. 2, this consists of an association of two representative positions corresponding to distance measurements performed by sensors at the time points t₅ and t₆. In the example illustrated in FIG. 2, there is therefore only one possible pair of associated representative positions (65, 75). Nonetheless, when the number of associated representative positions (65, 75) is greater than two, there are several ways for pairing associated representative positions, both in number and in pairs.

Afterwards, the processing unit (30) is configured to decide, when said speed is higher than a predetermined second value, that said person or an object has performed a movement in the room between the time points associated with the associated representative positions (65, 75) for which said speed has been calculated. The predetermined second value may be different for each association (100) of representative positions (65, 75, 85, 95). In general, the predetermined second value may be different for each speed calculated for a pair of representative positions (65, 75).

Preferably, the predetermined second value is comprised between 0 m/s and 2 m/s, more preferably between 0 m/s and 1 m/s. Even more preferably, the predetermined second value is 0.1 m/s.

In a preferred embodiment of the invention, the combination of the sensor distance measurements obtained at different time points is an average. Also, in accordance with this preferred embodiment, a combined distance measurement is obtained for a distance sensor by averaging distance measurements performed by said sensor at several time points.

In a preferred embodiment of the invention, the processing unit (30) is configured to:
form at least one association (100) of representative positions (65, 75, 85, 95) and select, within each of the formed associations of representative positions, at least one pair of representative positions (65, 75) and calculate, for each of the selected pairs of representative positions (65, 75), a speed as being the distance between the representative positions (65, 75) of the selected pair divided by the duration separating the subsequent time points (t₅, t₆) associated with the representative positions (65, 75) of said pair,
determine whether all of the speeds calculated for the selected pairs of representative positions are lower than the predetermined second value,
decide, if so is the case, that the person or the object has stopped all movements.

The pairs of representative positions may be formed in any manner, just as their number could vary. Thus, a representative position (65, 75, 85, 95) may be associated with several representative positions (65, 75, 85, 95) of the same association (100) of representative positions.

Preferably, speeds are determined by the processing unit (30) for pairs of representative positions formed by pairing each representative position of an association (100) of representative positions (65, 75, 85, 95) with the associated representative position following it directly chronologically and only with the latter. The chronology of the representative positions (65, 75, 85, 95) is given by the successive time points that are associated therewith (t₅, t₆, t₇, t₈).

FIG. 3 illustrates four selected parts of distance sensors (61, 71, 81, 91) corresponding to measurements performed by the sensors at four successive time points (t₅, t₆, t₇, t₈) and whose representative positions (65, 75, 85, 95) have been paired in accordance with this preferred embodiment of the invention, thereby providing, in the case of FIG. 3, three pairs of representative positions: (65, 75), (75, 85) and (85, 95).

Preferably, the successive time points (t₅, t₆, t₇, t₈) are separated by the same period.

In a preferred embodiment of the invention, the processing unit (30) is configured to:
determine whether the first representative position (65) is located in the first volume (1) or in a second volume (2) or in a third volume (3) in space, the first volume being a volume extending vertically upwards and/or downwards from a first horizontal surface of said room (for example the upper surface of the bed in this exemplary context), the second volume being a finite volume extending outwards from the lateral limits of the first volume, the third volume being a volume extending outwards from the lateral limits of the second volume,
decide that said person or object was located in that one amongst the first, second or third volume where said first representative position (65) is located at the subsequent time point associated therewith (t₅).

FIG. 1 shows an example of a first volume (1), a second volume (2) and a third volume (3), in dotted lines. Although this does not appear clearly in the figure, it should be understood that these three volumes are preferably exclusive, i.e. they have no points in common, possibly apart from their common border which, in the case of FIG. 1, is the external boundary of the first volume (1) and the external boundary of the second volume (2).

FIG. 1b is schematically shows a top view of the chamber of FIG. 1 which reveals the orthogonal projections of the first volume and of the second volume on the ground.

In a preferred embodiment of the invention, the processing unit (30) is configured to determine, or to interrogate another processing unit to know, whether said person is in his/her bed, has left his/her bed or has fallen after having left his/her bed. If the processing unit (30) is configured to interrogate another processing unit, it may consist of a processing unit of the same device or a processing unit of a third-party device. The logical interface between the two processing units may be standardised or proprietary, it may be in the form of an API or be a web service or a REST service or generally be a random one. The interrogation of a third-party processing unit may be carried out throughout a network, or an interconnection of networks, including throughout a public network like the Internet.

In several preferred embodiments of the invention which are introduced hereinafter, the processing unit (30) is configured to cross-reference different information concerning said person, in particular information relating to events having occurred to said person (like for example that he/she has just performed a movement or that he/she has just stopped all movements or that he/she has left his/her bed), whether this information is the result of a processing by the processing unit (30) or are obtained by interrogation of a third-party device. In these preferred embodiments of the invention, the processing unit (30) is also configured to deduce an event relating to said person from the cross-referencing of information.

In a preferred embodiment of the invention, the processing unit (30) is configured to verify at a given time point whether:
said person has left his/her bed and has not fallen,
said person or object has performed a movement in the room,
said person or object was located in the first volume (1) at the time of said movement,
   and to decide that said person has returned to his/her bed if all of these conditions are met simultaneously.

Preferably, the processing unit (30) cross-references this information each time a movement in the room has been detected.

In a preferred embodiment of the invention, the processing unit (30) is configured to verify at a given time point whether:
said person has left his/her bed and has not fallen,
said person or object has stopped all movements,
said person or object was last located in the first volume (1) or in the second volume (2),
   and to decide that said person has returned to his/her bed if all of these conditions are met simultaneously.

Preferably, the processing unit (30) cross-references this information each time said person or an object has stopped all movements.

In a preferred embodiment of the invention, the processing unit (30) is configured to verify at a given time point whether:
said person or an object has performed a movement,
said person was in bed before said movement,
said person or the object was located in the second volume (2) at the time of said movement,
   and to decide that said person has left his/her bed if all of these conditions are met simultaneously.

Preferably, the processing unit (30) cross-references this information each time said person or the object has performed a movement.

The present invention also relates to a method for detecting a movement of a person in a room including a bed, or an event relating to said person, the method comprising the following steps:
a. Setting up a detector (20) so that a detection field (50) of the detector covers at least one first volume (1) of said room and at least one portion of the environment of said first volume (1), said detector (20) including several distance sensors, each distance sensor being able to provide distance measurements (52) over time between said sensor and a corresponding obstacle in the line of sight (51) of said sensor, said detector (20) being connected to a processing unit (30) configured to process over time the distance measurements received from the detector,
b. calibrating the detector and the processing unit to define a space to be monitored in the detection field of the detector, this space to be monitored being divided into at least the first volume (1), a second volume (2) and a third volume (3), the first volume (1) being a volume extending vertically upwards and/or downwards from a first horizontal surface of said room, the second volume (2) being a volume extending outwards from lateral limits of the first volume (1), the third volume (3) being a volume extending outwards from the lateral limits of the second volume (2),
c. for each of the distance sensors of a first set of distance sensors of the detector (20), determining, by means of the processing unit (30), a first corresponding reference distance, either as a distance measured at a first time point (t₁) by the corresponding distance sensor, or as a combination of distances measured at several first time points (t₁, t₂, t₃, t₄) by the corresponding distance sensor,
d. determining, by means of the processing unit (30), a second set of distance sensors (60) which comprises those of the distance sensors of the first set including a distance measurement performed at a time point (t₅) subsequent to the first time point (t₁) or to the first time points (t₁, t₂, t₃, t₄) differs by more than a predetermined first value from the first reference distance of the corresponding distance sensor,
e. selecting, by means of the processing unit (30), at least one first part of the distance sensors (61) of the second set of distance sensors (60) and associating said subsequent time point (t₅) with the at least one first part of distance sensors (61),
f. determining, by means of the processing unit (30), for the selected at least one first part of distance sensors (61), a first representative position (65) of the positions in the space of the room of the obstacles corresponding to the distance sensors of said selected at least one first part of sensors (61), and associating with said first representative position (65) the subsequent time point (t₅),
g. repeating, by means of the processing unit (30) steps c. to f. at several other time points (t₆, t₇, t₈) subsequent to the first time point or to the several first time points,
h. associating, by means of the processing unit (30), representative positions (65, 75, 85, 95) associated with several different subsequent time points (t₅, t₆, t₇, t₈) to form a first association of representative positions (100),
i. selecting, by means of the processing unit (30), within the first association (100) of representative positions (65, 75, 85, 95), at least one first pair of representative positions (65, 75) and calculating a first speed as the distance between the representative positions (65, 75) of said first pair divided by the duration separating the time points (t₅, t₆) associated with the representative positions (65, 75) of said first pair,
j. deciding, by means of the processing unit (30), when said first speed is higher than a predetermined second value, that the person or the object has performed a movement in the room between the time points (t₅, t₆) associated with the representative positions (65, 75) of said first pair of representative positions,
k. if so is not the case, forming, by means of the processing unit (30), at least one association (100) of representative positions (65, 75, 85, 95) and selecting, within each of the formed associations of representative positions, at least one pair of representative positions (65, 75) and calculating, for each of the selected pairs of representative positions (65, 75), a speed as being the distance between the representative positions (65, 75) of the selected pair divided by the duration separating the subsequent time points (t₅, t₆) associated with the representative positions (65, 75) of said pair and determining that all the speeds calculated for the selected pairs of representative positions are lower than the predetermined second value,
l. deciding, if so is the case, by means of the processing unit (30), that said person or the object has stopped all movements.

Preferably, the different steps of the method are executed at regular time intervals, preferably each time new distance measurements are received from sensors.

In a preferred embodiment of the invention, the method comprises the following steps:
a. If said person or an object has performed a movement in the room, by means of the processing unit (30), determining, or interrogating another processing unit to know, whether, at the time of said movement, said person has left the first volume (1) of said room and has not fallen,
b. if so is the case, by means of the processing unit (30), determining whether the last detected movement of said person or of an object was in the first volume (1),
c. if so is the case, deciding, by means of the processing unit (30), that said person has returned to the first volume (1).

In a preferred embodiment of the invention, the method comprises the following steps:
a. if said person or an object has stopped all movements, by means of the processing unit (30), determining, or interrogating another processing unit to know, whether, at the time of stopping all movements, said person has left the first volume (1) and has not fallen,
b. if so is the case, determining, by means of the processing unit (30), whether the last detected movement of said person or of the object was in the first volume (1) or in the second volume (2),
c. if so is the case, deciding, by means of the processing unit (30), that said person has returned to the first volume (1).

The present invention has been described in connection with specific embodiments, which have a purely illustrative value and should not be considered as restrictive. In general, it is obvious to a person skilled in the art that the present invention is not limited to the examples illustrated and/or described hereinabove. The presence of reference numerals in the drawings should not be considered as restrictive, including when these numerals are indicated in the claims.

The use of the verbs “comprise”, “include”, or any other variation, as well as their conjugations, cannot in any way exclude the presence of sensors other than those mentioned.

The use of the indefinite article “a”, “an”, or of the definite article “the”, to introduce a sensor does not exclude the presence of a plurality of these sensors.

The invention may also be described as follows: a device (10) and a method for detecting whether a person or an object located in a room has performed a movement or has interrupted a movement, and being able to deduce therefrom whether an event has happened to said person like, for example, that he/she has left an area of the room or that he/she has returned to it after having left it. The device performs an analysis over time of the distances between the sensors of a detector (20) and the obstacles present in the detection field (50) of the detector. The detection of a movement or of the interruption of a movement is based on the analysis of the variations of said distances over time. The deduction of an event is based on the location of the obstacles moving within a space (60) of the room and on the history of the events that have occurred to said person within the room.

1. A device for detecting a movement or a stop of a movement of a person or an object in a room, or an event relating to said person, the device comprising:
a detector having a detection field able to cover at least one first volume of said room and at least one portion of the environment of the first volume, said detector including several distance sensors, each distance sensor having a line of sight and being able to provide distance measurements over time between said sensor and a corresponding obstacle, i.e. an obstacle located in the line of sight of said sensor,
a processing unit connected to the detector and configured to process over time the distance measurements received from the distance sensors of the detector,
characterised in that the processing unit is configured to perform the following steps and in that order:
a. for each of the distance sensors of a first set of distance sensors of the detector, determining a first corresponding reference distance either as a distance measured at a first time point by the corresponding distance sensor, or as being a combination of distances measured at several first time points by the corresponding distance sensor,
b. determining a second set of distance sensors which consists of those of the distance sensors of the first set including a distance measurement performed at a time point subsequent to the first time point or to the first time points differs by more than a predetermined first value from the first reference distance of the corresponding distance sensor,
c. selecting at least one first part of the distance sensors of the second set of distance sensors and associating said subsequent time point with the at least one first part of distance sensors,
d. determining, for the selected at least one first part of distance sensors, a first representative position of the positions in space of the obstacles corresponding to the distance sensors of said selected at least one first part of sensors, and associating the subsequent time point with said first representative position,
e. repeating steps a. to d. at several other time points subsequent to the first time point or to the first time point,
f. associating representative positions associated with the several different subsequent time points to form a first association of representative positions,
g. selecting, within the first association of representative positions, at least one first pair of representative positions and calculating a first speed as the distance between the representative positions of said first pair divided by the duration separating the subsequent time points associated with the representative positions of said first pair,
h. deciding, when said first speed is higher than a predetermined second value, that the person or the object has performed a movement in the room between the subsequent time points associated with the representative positions of said first pair of representative positions.

2. The device according to claim 1, characterised in that the combination of the distances measured at the first time points is an average of the distances measured at the first time points.

3. The device according to claim 1, characterised in that said first representative position is the position of the geometric centre of the obstacles corresponding to said selected at least one first part of distance sensors.

4. The device according to claim 1, characterised in that the processing unit is configured to:
form at least one association of representative positions and select, within each of the formed associations of representative positions, at least one pair of representative positions and calculate, for each of the selected pairs of representative positions, a speed as being the distance between the representative positions of the selected pair divided by the duration separating the subsequent time points associated with the representative positions of said pair,
determine whether all of the speeds calculated for the selected pairs of representative positions are lower than the predetermined second value,
decide, if so is the case, that the person or the object has stopped all movements.

5. The device according to claim 4, characterised in that the speeds are calculated for selected pairs of representative positions forming part of the same association of pairs of representative positions formed by pairing each representative position forming part of said association with a representative position of said association which directly follows it chronologically, and only with the latter.

6. The device according to claim 1, characterised in that the processing unit is configured to:
determine whether the first representative position is located in the first volume or in a second volume or in a third volume in space, the first volume being a volume extending vertically upwards and/or downwards from a first horizontal surface of said room, the second volume being a finite volume extending outwards from the lateral limits of the first volume, the third volume being a volume extending outwards from the lateral limits of the second volume,
decide that said person or object was located in that one amongst the first, second or third volume where said first representative position is located at the subsequent time point associated therewith.

7. The device according to claim 1, characterised in that the processing unit is configured to determine, or to interrogate another processing unit to know whether said person is in the first volume or has left the first volume or has fallen after having left the first volume.

8. The device according to claim 7, characterised in that the processing unit is configured to verify at a given time point whether:
said person has left the first volume and has not fallen,
said person or object has performed a movement in the room,
said person or object was located in the first volume at the time of said movement,
and to decide that said person has returned to the first volume if all of these three conditions are met simultaneously.

9. The device according to claim 7, characterised in that the processing unit is configured to verify at a given time point whether:
said person has left the first volume and has not fallen,
said person or object has stopped all movements,
said person or object was last located in the first volume or in the second volume,
and to decide that said person has returned to the first volume if all of these three conditions are met simultaneously.

10. The device according to claim 7, characterised in that the processing unit is configured to verify at a given time point whether:
said person or an object has performed a movement,
said person was in bed before said movement,
said person or an object was located in the second volume at the time of said movement,
and to decide that said person has left the first volume if all of these conditions are met simultaneously.

11. A method for detecting a movement or a stop of a movement of a person or object in a room, or an event relating to said person, the method comprising the following steps:
a. setting up a detector so that a detection field of the detector covers at least one first volume of said room and at least one portion of the environment of said first volume, said detector including several distance sensors, each distance sensor being able to provide distance measurements over time between said sensor and a corresponding obstacle in the line of sight of said sensor, said detector being connected to a processing unit configured to process over time the distance measurements received from the detector,
b. calibrating the detector and the processing unit to define a space to be monitored in the detection field of the detector, this space to be monitored being divided into at least the first volume, a second volume and a third volume, the first volume being a volume extending vertically upwards and/or downwards from a first horizontal surface of said room, the second volume being a volume extending outwards from lateral limits of the first volume, the third volume being a volume extending outwards from the lateral limits of the second volume,
c. for each of the distance sensors of a first set of distance sensors of the detector, determining, by means of the processing unit, a first corresponding reference distance, either as a distance measured at a first time point by the corresponding distance sensor, or as a combination of distances measured at several first time points by the corresponding distance sensor,
d. determining, by means of the processing unit, a second set of distance sensors which comprises those of the distance sensors of the first set including a distance measurement performed at a time point subsequent to the first time point or to the first time points differs by more than a predetermined first value from the first reference distance of the corresponding distance sensor,
e. selecting, by means of the processing unit, at least one first part of the distance sensors of the second set of distance sensors and associating said subsequent time point with the at least one first part of distance sensors,
f. determining, by means of the processing unit, for the selected at least one first part of distance sensors, a first representative position of the positions in the space of the room of the obstacles corresponding to the distance sensors of said selected at least one first part of sensors, and associating with said first representative position the subsequent time point,
g. repeating, by means of the processing unit, steps c. to f. at several other time points subsequent to the first time point or to the several first time points,
h. associating, by means of the processing unit, representative positions associated with several different subsequent time points to form a first association of representative positions,
i. selecting, by means of the processing unit, within the first association of representative positions, at least one first pair of representative positions and calculating a first speed as the distance between the representative positions of said first pair divided by the duration separating the time points associated with the representative positions of said first pair,
j. deciding, by means of the processing unit, when said first speed is higher than a predetermined second value, that the person or the object has performed a movement in the room between the time points associated with the representative positions of said first pair of representative positions,
k. if so is not the case, forming, by means of the processing unit, at least one association of representative positions and selecting, within each of the formed associations of representative positions, at least one pair of representative positions and calculating, for each of the selected pairs of representative positions, a speed as being the distance between the representative positions of the selected pair divided by the duration separating the subsequent time points associated with the representative positions of said pair and determining that all the speeds calculated for the selected pairs of representative positions are lower than the predetermined second value,
l. deciding, if so is the case, by means of the processing unit, that said person or the object has stopped all movements.

12. The method according to claim 11, comprising the following steps:
If said person or an object has performed a movement in the room, by means of the processing unit, determining, or interrogating another processing unit to know, whether, at the time of said movement, said person has left the first volume of said room and has not fallen,
if so is the case, by means of the processing unit, determining whether the last detected movement of said person or of an object was in the first volume,
if so is the case, deciding, by means of the processing unit, that said person has returned to the first volume.

13. The method according to claim 11, comprising the following steps:
if said person or an object has stopped all movements, by means of the processing unit, determining, or interrogating another processing unit to know, whether, at the time of stopping all movements, said person has left the first volume and has not fallen,
if so is the case, determining, by means of the processing unit, whether the last detected movement of said person or of the object was in the first volume or in the second volume,
if so is the case, deciding, by means of the processing unit, that said person has returned to the first volume.
